(19) United States
(12) Patent Application Publication
(10) Pub. No.: US 2014/0276392 A1
(43) Pub. Date: Sep. 18, 2014

(54) INPUT DEVICE FOR ROBOTIC CATHETER AND GUIDE WIRE SYSTEM

(71) Applicant: Hansen Medical, Inc., Mountain View, CA (US)

(72) Inventors: Serena Wong, Mountain View, CA (US); Richard Henderson, Fremont, CA (US); June Park, Palo Alto, CA (US); Sean Walker, Fremont, CA (US); Kamini Balaji, Mountain View, CA (US); Jason Hsu, Mountain View, CA (US)

(73) Assignee: Hansen Medical, Inc., Mountain View, CA (US)

(21) Appl. No.: 13/833,074

(22) Filed: Mar. 15, 2013

(51) Int. Cl. A61M 25/01

(52) U.S. Cl. CPC A61M 25/0105
USPC 604/95.01

(57) ABSTRACT

An input device for a robotic catheter system includes an inner member having a generally cylindrical shape and defining an axis, and an outer member coaxially disposed on the inner member and configured to rotate about and move along the axis defined by the inner member to control a position and orientation of at least one of a catheter and a guide wire. Rotating the outer member about the axis causes the catheter, the guide wire, or both, to rotate and wherein moving the outer member along the axis causes the catheter, the guide wire, or both, to advance or retract relative to a patient's body.

### BACKGROUND

Robotically controlled catheter systems allow clinicians to direct catheters to various locations within a patient's body. Once in place, the catheter can be manipulated to treat various diseases or help a clinician perform various surgical procedures. For instance, balloon catheters may be used during an angioplasty procedure to widen or clear obstructed arteries. Other types of catheters may be used to administer drugs to a patient or to facilitate the draining of bodily fluids (e.g., a Foley catheter).

### SUMMARY

An exemplary input device includes an inner member that defines an axis and an outer member coaxially disposed on the inner member. The outer member is configured to rotate about and move along the axis defined by the inner member to control a position and orientation of at least one of a catheter and a guide wire. Rotating the outer member about the axis causes the catheter, the guide wire, or both, to rotate. Moving the outer member along the axis causes the catheter and guide wire to advance or retract relative to a patient's body.

An exemplary system includes an actuator and an input device. The actuator is configured to manipulate a position and orientation of a catheter assembly, which includes a catheter and a guide wire. The input device has an inner member that defines an axis and an outer member that is coaxially disposed on the inner member and configured to rotate about and move along the axis defined by the inner member. The movement of the outer member can be used to control the position and orientation of at least one of the catheter and the guide wire. Rotating the outer member about the axis causes at least one of the catheter and the guide wire to rotate. Moving the outer member along the axis causes the catheter and the guide wire to advance or retract within the patient's body.

A robotic catheter system includes a catheter assembly, an actuator, and an input device. The catheter assembly includes a catheter and a guide wire disposed within the catheter. The actuator is configured to manipulate a position and orientation of the catheter assembly. The input device has an inner member defining an axis and an outer member coaxially disposed on the inner member. The outer member is configured to rotate about and move along the axis defined by the inner member to control the position and orientation of at least one of the catheter and the guide wire. The inner member has a generally cylindrical shape, and rotating the outer member about the axis cause the catheter assembly to rotate. Moving the outer member along the axis causes the catheter assembly to advance or retract relative to the patient's body. The input device includes a position detector configured to determine a linear position of the outer member relative to the inner member and an angular position of the outer member relative to the inner member and output at least one position signal representing at least one of the linear position and angular position of the outer member relative to the inner member.

### BRIEF DESCRIPTION OF THE DRAWINGS

FIG. 1 illustrates an exemplary system for manipulating the position and orientation of a catheter assembly in a patient's body.

FIG. 2 illustrates exemplary components of an input device for manipulating the position and orientation of the catheter assembly.

FIG. 3 illustrates an input device having an exemplary position detector with buttons and an encoder.

FIG. 4 illustrates another exemplary position detector having buttons and an encoder.

FIG. 5 illustrates an exemplary position detector providing a restorative force.

FIG. 6 illustrates another exemplary position detector providing a restorative force.

FIG. 7 illustrates an input device having a motor for manipulating the position and orientation of the catheter assembly.

FIG. 8 illustrates an input device using an encoder for rotation of the catheter assembly.

FIG. 9 illustrates another input device using an encoder for rotation of the catheter assembly.

FIG. 10 illustrates an input device using buttons to rotate the catheter assembly.

FIG. 11 illustrates an input device having a ball joint for providing an additional degree of freedom for manipulating the position and orientation of the catheter assembly.

### DETAILED DESCRIPTION

An exemplary input device for a robotic catheter system includes an inner member defining an axis and an outer member coaxially disposed on the inner member. The outer member rotates about and moves along the axis defined by the inner member to control a position and orientation of a catheter, a guide wire, or both as part of a catheter assembly. The inner member has a generally cylindrical shape, and rotating the outer member about the axis defined by the inner member causes the catheter assembly to rotate. Moving the outer member along the axis causes the catheter assembly to advance or retract relative to the patient's body.

The robotic catheter system may further include an actuator that manipulates a position and orientation of the catheter assembly while the catheter assembly is inside the patient's body. In some implementations, the input device includes a position detector that determines a linear position, an angular position, or both, of the outer member relative to the inner member. The position detector outputs one or more position signals representing the detected position.

During use of the robotically controlled catheter system, a patient may be positioned on an operating table or surgical bed. A clinician can monitor the procedure and control the catheter assembly via a workstation, and in particular, the input device located at the workstation. The components of the robotically controlled catheter system may be in communication with one another via a plurality of cables or other connectors that can provide data communication. In some exemplary approaches, the components may communicate wirelessly.

The Figures illustrate exemplary components of a system for manipulating the position and orientation of a catheter assembly. The system may take many different forms and include multiple and/or alternate components and facilities. While an exemplary system is shown, the exemplary components illustrated are not intended to be limiting. Indeed, additional or alternative components and/or implementations may be used. Moreover, some components illustrated in the Figures have been simplified for purposes of clarity. Therefore, the components are not necessarily drawn to scale and certain aspects of some component may be omitted.

As illustrated in FIG. 1, the system 100 includes a catheter assembly 105, an actuator 110, and an input device 115.

The catheter assembly 105 may include a catheter 120 and a guide wire 125. The catheter 120 may include a generally hollow tube having sufficient flexibility to travel through a patient's body during, e.g., surgical procedures or other medical treatments. Different types of catheters 120 may be configured to travel through different parts of the patient's body. For instance, a catheter 120 for performing angioplasty procedures may have a different size and flexibility than a catheter 120 used to administer drugs or drain bodily fluids. The catheter 120 may also carry any number of medical instruments (not shown) such as a balloon, stent, or physiological sensors.

The guide wire 125 may be disposed within the catheter 120 and configured to facilitate movement of the catheter 120 through the patient's body. The catheter 120 and guide wire 125 may move through the patient's body together or the catheter 120 and guide wire 125 may move independently of one another. For instance, the catheter 120 and guide wire 125 may be inserted together into the patient's body until the catheter assembly 105 reaches a surgical site. Once positioned, the guide wire 125 may be removed and the catheter 120 may remain to deploy any medical instruments carried by the catheter.

The components of the catheter assembly 105 may be manipulated as the catheter assembly 105 moves through the patient's body. As used in the following discussion, the term “advance” may refer to pushing the catheter assembly 105, which may cause any part of the catheter assembly 105 to move further into a patient's body, and the term “retract” may refer to pulling the catheter assembly 105, which may cause any part of the catheter assembly 105 to be removed from the patient's body. Portions of the catheter assembly 105 may be configured to bend relative to other portions. For instance, the tip of the catheter 120, guide wire 125, or both, may be configured to bend relative to the body of the catheter 120, guide wire 125, or both. The catheter assembly 105 may be further configured to rotate, as discussed below.

The actuator 110 may include any device configured to facilitate the movement of the catheter assembly 105 through the patient's body. The actuator 110 may be configured to cause the catheter assembly 105 to advance or retract relative to the patient's body. Moreover, the actuator 110 may cause the catheter assembly 105 to rotate or for portions of the catheter assembly 105 to bend relative to other portions. The actuator 110 may include any number of components configured to manipulate the position and orientation of the components of the catheter assembly 105. In one possible implementation, the actuator 110 may be configured to receive control signals from, e.g., the input device 115, and manipulate the position and orientation of the components of the catheter assembly 105 accordingly. For instance, the actuator 110 may be configured to receive an advance signal and push the catheter 120, the guide wire 125, or both, further into the patient's body in accordance with the advance signal. The actuator 110 may be configured to receive a retract signal and pull at least part of the catheter 120, the guide wire 125, or both, from the patient's body in accordance with the retract signal. The actuator 110 may be configured to receive a rotate signal and rotate the catheter 120, the guide wire 125, or both, in accordance with the rotate signal. The actuator 110 may include any number of components (not shown) to push, pull, and rotate the components of the catheter assembly 105. For instance, one or more motors (not shown) may be configured to feed (i.e., push) the catheter assembly 105 and the same or different motors may be configured to pull the catheter assembly 105 from the patient. Moreover, the actuator 110 may include wires (not shown) connected to various portions of the catheter assembly 105 that when pulled, cause portions of the catheter assembly 105 to bend in various directions. The actuator 110 may include motors that wind the wires to change the distance between the motor and the portion of the catheter assembly 105 to which the wire is connected. Separate motors may control each wire, thus allowing the actuator 110 to manipulate different parts of the catheter assembly 105 independently.

The input device 115 may be configured to allow a clinician 130 or other medical personnel to control the position and orientation of the catheter assembly 105 within the patient. The input device 115 may be configured to receive an input from the clinician 130 based on the way the clinician 130 physically manipulates the position of the input device 115. As shown, the input device 115 provides the clinician 130 with multiple degrees of freedom, each associated with a different movement of the catheter assembly 105, so that the clinician 130 can control the catheter assembly 105 as if the clinician 130 were manipulating the position and orientation of the catheter assembly 105 directly.

The input device 115 may include a processor 135 configured to interpret the input from the clinician 130 and generate and output corresponding signals to the actuator 110. For clarity, the processor 135 is shown outside the input device 115. In some possible approaches, however, the processor 135 may be embedded in the input device 115. The processor 135 may be configured to generate an advance signal when the clinician 130 indicates a desire to push the catheter assembly 105 into the patient's body. The processor 135 may be further configured to generate a retract signal when the clinician 130 indicates a desire to pull at least a portion of the catheter assembly 105 from the patient's body. Moreover, the processor 135 may be configured to generate a rotate signal when the clinician 130 indicates a desire to rotate the catheter assembly 105. As discussed above, the clinician's 130 desire for controlling the catheter assembly 105 may be expressed through movement of the input device 115. The processor 135 may interpret these movements based on the outputs of various sensors of the input device 115. FIGS. 2-11 illustrate exemplary components, including various sensors, of the input device 115 that are configured to detect the movements of the input device 115 that the processor 135 may use to determine the clinician's 130 desired manipulation of the catheter assembly 105.

FIG. 2 illustrates exemplary components of the input device 115 for manipulating the position and orientation of the catheter assembly 105. As shown in FIG. 2, the input device 115 includes an inner member 140 and an outer member 145. The inner member 140, as illustrated, has a generally cylindrical shape that defines an axis 150. The outer member 145 is coaxially disposed on the inner member 140 and is configured to rotate about and move along the axis 150. As discussed in greater detail below, this movement of the outer member 145 relative to the inner member 140 may be detected and signals representing the movement may be output to the processor 135 for the processor 135 to determine the clinician's 130 desired operation of the catheter assembly 105. Specifically, rotating the outer member 145 about the axis 150 may indicate the clinician's 130 desire to rotate the catheter 120, the guide wire 125, or both. Moving the outer member 145 along the axis 150 may indicate the clinician's 130 desire to advance (e.g., push) or retract (e.g., pull) the catheter 120, the guide wire 125, or both relative to the patient's body.

Referring to FIG. 3, the input device 115 includes a position detector 155 with buttons 160A, 160B (collectively, 160) and an encoder 165 configured to detect one or more positions of a flange 170 that may be disposed on or integrally formed with the outer member 145. The position detector 155 may be configured to determine a position of the outer member 145 relative to the inner member 140 and output signals representing the position to the processor 135. The buttons 160 may include any type of mechanical or electrical switch that may be used to determine the position of the outer member 145 relative to the inner member 140. The position detector 155 of FIG. 3 includes a first button 160A configured to output an advance signal indicating the clinician's 130 desire to push the catheter assembly 105 into the patient's body. The clinician 130 may actuate the first button 160A by pushing the outer member 145 along the axis 150 until the flange 170 engages the first button 160A, thus placing the outer member 145 in a first position. When actuated by the flange 170, the first button 160A may be configured to generate and output the advance signal to the processor 135. The processor 135 may process and transmit the advance signal to the actuator 110 to cause the catheter assembly 105 to advance relative to the patient's body. The position detector 155 may further include a second button 160B configured to output a retract signal when actuated by the flange 170. The clinician 130 may actuate the second button 160B, therefore, by pulling the outer member 145 along the axis 150 to a second position.

The encoder 165 may be configured to output a signal representing one or more intermediate positions of the outer member 145 relative to the inner member 140. This way, the position detector 155 may be configured to detect other positions besides the first position and the second position. Any type of encoder 165 may be used. For instance, the encoder 165 may include an optical encoder, an inductive encoder, a capacitive encoder, a magnetic encoder, or the like. The encoder 165, illustrated as a linear encoder in FIG. 3, may be used to determine intermediate positions of the outer member 145 relative to the inner member 140 based on a position of the flange 170 relative to the encoder 165. The intermediate positions may be between the first position and the second position, discussed above. The encoder 165 may output a signal to the processor 135 that represents a magnitude of displacement from a predetermined reference point. The processor 135 may be configured to interpret the signal output by the encoder 165 to determine the intermediate position of the outer member 145. Furthermore, the output of the encoder 165 may be used to identify a problem with the input device 115, such as a failure of the first button 160A or second button 160B to register actuation by the flange 170. For instance, the processor 135 may be configured to identify a problem if the signal from the encoder 165 indicates that the outer member 145 is in the first position but no corresponding signal is received from the first button 160A. A similar methodology may be used to detect problems with the second button 160B.

The degree to which the clinician 130 pushes or pulls the outer member 145 relative to the inner member 140 may give the clinician 130 some control over the speed at which the catheter assembly 105 moves. As discussed above, the flange 170 may engage the first button 160A when the outer member 145 is in the first position and the flange 170 may engage the second button 160B when the outer member 145 is in the second position. The position detector 155 may output a signal representing the position of the outer member 145 to the processor 135, and the processor 135, in one possible implementation, may cause the actuator 110 to operate at a speed consistent with the position of the outer member 145. For instance, when in the first position or second position, the processor 135 may instruct the actuator 110 to move the catheter assembly 105 more quickly than when the outer member 145 is in an intermediate position, which as discussed above may include one or more positions between the first position and the second position. In some possible approaches, the processor 135 may cause the operating speed of the actuator 110 to increase as the flange 170, starting from a neutral position which may be one of the intermediate positions, approaches the first button 160A, the secondary button 160, or both.

FIG. 4 illustrates another exemplary position detector 155. In this possible implementation, the encoder 165 is disposed directly on the outer member 145 and is configured to detect a pattern 195 disposed on a housing (not shown) that surrounds the outer member 145. The pattern 195 may be disposed about the axis 150 so that the encoder 165 can read the pattern 195 regardless of the rotation of the outer member 145 relative to the inner member 140. The encoder 165 may be configured to determine the position, speed, angle of rotation, or any combination thereof, of the outer member 145 based on the pattern 195. The encoder 165 may be configured to output a signal to the processor 135 that causes the processor 135 to control the actuator 110 according to the detected position, whether linear or angular, or speed.

FIGS. 5-7 illustrate input devices 115 having exemplary position detectors 155 that provide a restorative force to the linear (see FIGS. 5-6) or rotational (see FIG. 7) motion of the outer member 145. The restorative force helps the outer member 145 return to a neutral position when the clinician 130, for instance, releases his or her hand from the input device 115. As shown in FIG. 5, the position detector 155 may include one or more biasing devices 175, illustrated as springs 180, to bias the outer member 145 to a neutral position, which may be located between the first position and the second position, relative to the inner member 140. When the outer member 145 is in the neutral position, the actuator 110 may be configured to maintain the position and orientation of the catheter assembly 105. In other words, the actuator 110 may neither advance, retract, nor rotate any part of the catheter assembly 105 while the outer member 145 is in the neutral position. In some possible implementations, the biasing device 175 may simply prevent the outer member 145 from staying in the first or second positions when the clinician 130 lifts his or her hands from the input device 115. In such instances, the biasing devices 175 may push the outer member 145 to an intermediate position that allows for some movement (e.g., advancement, retraction, or rotation) of the catheter assembly 105 but at a slower speed than if the outer member 145 were in the first position or the second position.

In the embodiment of FIG. 6, the biasing device 175 includes a spring 180 and a motor 185 having a rotating output shaft 190. The output shaft 190 of the motor 185 may be configured to engage the flange 170 such that the rotation of the output shaft 190 may drive the flange 170 either toward or away from the motor 185. In one possible approach, the output shaft 190 may include external threads and the flange 170 may include internal threads configured to receive the threads of the output shaft 190. The motor 185 may be configured to return the outer member 145 to the neutral position when the clinician 130 removes his or her hand from the input device 115. That is, the motor 185 may generally allow the outer member 145 to freely move along the axis 150 or rotate about the axis 150 when the outer member 145 is manipulated by the clinician 130. When the clinician 130 removes his or her hand from the input device 115, however, the motor 185 may be configured to bias the outer member 145 back to the neutral position until, e.g., the clinician 130 resumes control of the input device 115. Thus, the motor 185 may hold the outer member 145 in the neutral position when the input device 115 is not in use.

FIG. 7 illustrates an exemplary implementation where the input device 115 includes a motor 185 configured to provide a restorative force following rotation of the outer member 145. In FIG. 7, the motor 185 is disposed on an end of the inner member 140. Generally, the motor 185 may be configured to allow the outer member 145 to freely rotate about the axis 150. When the clinician 130 releases the input device 115, however, the motor 185 may be configured to return the outer member 145 to a neutral position where no rotation of the catheter assembly 105 is commanded by the processor 135. One way for the motor 185 to return the outer member 145 to the neutral position is to move the inner member 140. Alternative approaches may place the motor 185 on the outer member 145.

FIGS. 8-9 illustrate exemplary input devices 115 that use an encoder 165 for controlling rotation of the catheter assembly 105. Referring to FIG. 8, the encoder 165 is disposed on the inner member 140 and a pattern 195 is printed on an inside surface of the outer member 145. As the outer member 145 rotates relative to the inner member 140, the encoder 165 determines an angular position of the outer member 145 relative to the inner member 140. The encoder 165 generates a signal representing the angular position and outputs the signal to the processor 135 so that the processor 135 may generate the appropriate command signal for the actuator 110 to cause the catheter assembly 105 to rotate according to the angular position. In the possible implementation illustrated in FIG. 9, the pattern 195 may be printed on an outer surface of the outer member 145 and the encoder 165 may be spaced from the outer member 145 and the inner member 140. The encoder 165 shown in FIG. 9 may be configured to determine the angular position of the outer member 145 by reading the pattern 195 printed on the outer member 145. The processor 135 may control the operation of the actuator 110, which in turn may control the rotation of the catheter assembly 105, according to the angular position detected by the encoder 165.

FIG. 10 illustrates one possible implementation where the input device 115 includes buttons to rotate the catheter assembly 105. The buttons may include a first button 200 and a second button 205 disposed on the outer member 145 and a first flange 210 and a second flange 215 disposed on the inner member 140. The outer member 145 may be configured to rotate, upon actuation by the clinician 130, such that the first flange 210 engages the first button 200 and the second flange 215 engages the second button 205, commanding the catheter assembly 105 to rotate in a first direction (e.g., clockwise). Another possible manipulation from the clinician 130 may cause the first flange 210 to engage the second button 205 and the second flange 215 to engage the first button 200, which may command the catheter assembly 105 to rotate in a second direction (e.g., counter-clockwise). The first button 200 and the second button 205 may be configured to output signals representing actuation by the first flange 210 or the second flange 215 to the processor 135, which may generate the control signals to control, via the actuator 110, the rotation of one or more components of the catheter assembly 105 accordingly. Each button 200, 205 may output a signal indicating which flange 210, 215 has engaged the button since each flange 210, 215 will engage different sides of each button 210, 215. The processor 135 may, therefore, be configured to determine whether the buttons 200, 205 were engaged as a result of the clinician 130 turning the outer member 145 clockwise or counter-clockwise.

With the first flange 210, the second flange 215, the first button 200, and the second button 205, the processor 135 may be further configured to identify problems with, e.g., one of the first button 200 and the second button 205. For instance, the first flange 210 and second flange 215 may be located such that the first button 200 engages either the first flange 210 or the second flange 215 and the second button 205 engages the other. Thus, when functioning properly, the processor 135 will receive signals indicating that the first button 200 has been engaged by one flange and the second button 205 has been engaged by the other. If the processor 135 receives conflicting signals (e.g., signals that indicate only one button 200, 205 has engaged one flange 210, 215), the processor 135 can identify a problem with one of the buttons 200, 205. Moreover, because the first and second buttons 200, 205 are actuated by the first and second flanges 210, 215 from different sides of each button, the processor 135 may be configured to determine if an entire button has malfunctioned or just one side of the button.

FIG. 11 illustrates an input device 115 having a ball joint 220 for providing the clinician 130 with an additional degree of freedom for manipulating the position and orientation of the catheter assembly 105. The ball joint 220 may be located at a distal end of the inner member 140 to allow the outer member 145 and inner member 140 to rotate about an axis 225 defined by the ball joint 220. Moreover, the ball joint 220 may allow for rotation of the inner and outer members 140, 145 about the axis 150 defined by the inner member 140. The input device 115 may include one or more sensors (not shown) such as an accelerometer, a gyroscope, or both, configured to detect movement and output signals to the processor 135 so that the processor 135 may control the actuator 110 in accordance with the signals received.

In general, computing systems and/or devices, such as the processor may employ any of a number of computer operating systems, including, but by no means limited to, versions and/or varieties of the Microsoft Windows® operating system, the Unix operating system (e.g., the Solaris® operating system distributed by Oracle Corporation of Redwood Shores, Calif.), the AIX UNIX operating system distributed by International Business Machines of Armonk, N.Y., the Linux operating system, and the Mac OS X operating system distributed by Apple Inc. of Cupertino, Calif. Examples of computing devices include, without limitation, a computer workstation, a server, a desktop, notebook, laptop, or handheld computer, or some other computing system and/or device.

Computing devices generally include computer-executable instructions, where the instructions may be executable by one or more computing devices such as those listed above. Computer-executable instructions may be compiled or interpreted from computer programs created using a variety of programming languages and/or technologies, including, without limitation, and either alone or in combination, Java™, C, C++, Visual Basic, Java Script, Perl, etc. In general, a processor (e.g., a microprocessor) receives instructions, e.g., from a memory, a computer-readable medium, etc., and executes these instructions, thereby performing one or more processes, including one or more of the processes described herein. Such instructions and other data may be stored and transmitted using a variety of computer-readable media.

A computer-readable medium (also referred to as a processor-readable medium) includes any non-transitory (e.g., tangible) medium that participates in providing data (e.g., instructions) that may be read by a computer (e.g., by a processor of a computer). Such a medium may take many forms, including, but not limited to, non-volatile media and volatile media. Non-volatile media may include, for example, optical or magnetic disks and other persistent memory. Volatile media may include, for example, dynamic random access memory (DRAM), which typically constitutes a main memory. Such instructions may be transmitted by one or more transmission media, including coaxial cables, copper wire and fiber optics, including the wires that comprise a system bus coupled to a processor of a computer. Common forms of computer-readable media include, for example, a floppy disk, a flexible disk, hard disk, magnetic tape, any other magnetic medium, a CD-ROM, DVD, any other optical medium, punch cards, paper tape, any other physical medium with patterns of holes, a RAM, a PROM, an EPROM, a FLASH-EEPROM, any other memory chip or cartridge, or any other medium from which a computer can read.

Databases, data repositories or other data stores described herein may include various kinds of mechanisms for storing, accessing, and retrieving various kinds of data, including a hierarchical database, a set of files in a file system, an application database in a proprietary format, a relational database management system (RDBMS), etc. Each such data store is generally included within a computing device employing a computer operating system such as one of those mentioned above, and are accessed via a network in any one or more of a variety of manners. A file system may be accessible from a computer operating system, and may include files stored in various formats. An RDBMS generally employs the Structured Query Language (SQL) in addition to a language for creating, storing, editing, and executing stored procedures, such as the PL/SQL language mentioned above.

In some examples, system elements may be implemented as computer-readable instructions (e.g., software) on one or more computing devices (e.g., servers, personal computers, etc.), stored on computer readable media associated therewith (e.g., disks, memories, etc.). A computer program product may comprise such instructions stored on computer readable media for carrying out the functions described herein.

With regard to the processes, systems, methods, heuristics, etc. described herein, it should be understood that, although the steps of such processes, etc. have been described as occurring according to a certain ordered sequence, such processes could be practiced with the described steps performed in an order other than the order described herein. It further should be understood that certain steps could be performed simultaneously, that other steps could be added, or that certain steps described herein could be omitted. In other words, the descriptions of processes herein are provided for the purpose of illustrating certain embodiments, and should in no way be construed so as to limit the claims.

Accordingly, it is to be understood that the above description is intended to be illustrative and not restrictive. Many embodiments and applications other than the examples provided would be apparent upon reading the above description. The scope should be determined, not with reference to the above description, but should instead be determined with reference to the appended claims, along with the full scope of equivalents to which such claims are entitled. It is anticipated and intended that future developments will occur in the technologies discussed herein, and that the disclosed systems and methods will be incorporated into such future embodiments. In sum, it should be understood that the application is capable of modification and variation.

All terms used in the claims are intended to be given their broadest reasonable constructions and their ordinary meanings as understood by those knowledgeable in the technologies described herein unless an explicit indication to the contrary in made herein. In particular, use of the singular articles such as “a,” “the,” “said,” etc. should be read to recite one or more of the indicated elements unless a claim recites an explicit limitation to the contrary.

The Abstract of the Disclosure is provided to allow the reader to quickly ascertain the nature of the technical disclosure. It is submitted with the understanding that it will not be used to interpret or limit the scope or meaning of the claims. In addition, in the foregoing Detailed Description, it can be seen that various features are grouped together in various embodiments for the purpose of streamlining the disclosure. This method of disclosure is not to be interpreted as reflecting an intention that the claimed embodiments require more features than are expressly recited in each claim. Rather, as the following claims reflect, inventive subject matter lies in less than all features of a single disclosed embodiment. Thus the following claims are hereby incorporated into the Detailed Description, with each claim standing on its own as a separately claimed subject matter.

1. A device comprising:
an inner member having a generally cylindrical shape and defining an axis;
an outer member coaxially disposed on the inner member and configured to rotate about and move along the axis defined by the inner member to control a position and orientation of at least one of a catheter and a guide wire,
wherein rotating the outer member about the axis causes the catheter and guide wire to rotate and wherein moving the outer member along the axis causes at least one of the catheter and guide wire to advance or retract relative to a patient's body.

2. The device of claim 1, further comprising a position detector configured to determine a position of the outer member relative to the inner member.

3. The device of claim 2, wherein the position detector includes:
a first button configured to output an advance signal indicating that the outer member is in a first position, the advance signal causing at least one of the catheter and the guide wire to advance relative to the patient's body; and
a second button configured to output a retract signal indicating that the outer member is in a second position, the retract signal causing at least one of the catheter and the guide wire to retract relative to the patient's body.

4. The device of claim 3, wherein the outer member includes a flange configured to engage the first button when the outer member is in the first position and the second button when the outer member is in the second position.

5. The device of claim 2, wherein the position detector is configured to output a position signal representing a position of the outer member between the first position and the second position.

6. The device of claim 5, wherein the position detector includes a linear encoder configured to determine the position of the outer member between the first position and the second position.

7. The device of claim 2, wherein the position detector includes a biasing device configured to bias the outer member toward a neutral position.

8. The device of claim 2, wherein the position detector includes an encoder configured to determine an angular position of the outer member relative to the inner member.

9. The device of claim 2, wherein the outer member includes a first button and a second button and wherein the inner member includes a first flange configured to engage the first button to cause the catheter to rotate in a first direction and configured to engage the second button to cause the catheter to rotate in a second direction.

10. The device of claim 9, wherein the outer member includes a second flange configured to engage the second button when the first flange engages the first button and configured to engage the first button when the first flange engages the second button.

11. The device of claim 1, further comprising a ball joint disposed at a distal end of the inner member and configured to allow the outer member and inner member to rotate about an axis defined by the ball joint.

12. A system comprising:
an actuator configured to manipulate a position and orientation of a catheter assembly having a catheter and a guide wire; and
an input device having an inner member having a generally cylindrical shape and defining an axis and an outer member coaxially disposed on the inner member and configured to rotate about and move along the axis defined by the inner member to control the position and orientation of at least one of the catheter and the guide wire, and
wherein rotating the outer member about the axis causes at least one of the catheter and the guide wire to rotate and wherein moving the outer member along the axis causes at least one of the catheter and the guide wire to advance or retract within the patient's body.

13. The system of claim 12, wherein the input device further includes a position detector configured to determine a position of the outer member relative to the inner member, wherein the position detector includes:
a first button configured to output an advance signal indicating that the outer member is in a first position, the advance signal causing the catheter assembly to advance relative to the patient's body; and
a second button configured to output a retract signal indicating that the outer member is in a second position, the retract signal causing the catheter assembly to retract.

14. The system of claim 13, wherein the outer member is configured to engage the first button when the outer member is in the first position and engage the second button when the outer member is in the second position.

15. The system of claim 13, wherein the position detector is configured to output a position signal representing a position of the outer member between the first position and the second position.

16. The system of claim 13, wherein the position detector includes a biasing device configured to bias the outer member toward a neutral position.

17. The system of claim 13, wherein the position detector includes an encoder configured to determine an angular position of the outer member relative to the inner member.

18. The system of claim 12, wherein the input device includes a ball joint disposed at a distal end of the inner member and configured to allow the outer member and inner member to rotate about an axis defined by the ball joint.

19. A robotic catheter system comprising:
a catheter assembly including a catheter and a guide wire disposed within the catheter;
an actuator configured to manipulate a position and orientation of the catheter assembly; and
an input device having an inner member defining an axis and an outer member coaxially disposed on the inner member and configured to rotate about and move along the axis defined by the inner member to control the position and orientation of at least one of the catheter and the guide wire, wherein the inner member has a generally cylindrical shape, and wherein rotating the outer member about the axis causes at least one of the catheter and the guide wire to rotate and wherein moving the outer member along the axis causes at least one of the catheter and the guide wire to advance or retract relative to the patient's body,
wherein the input device includes a position detector configured to determine a linear position of the outer member relative to the inner member and an angular position of the outer member relative to the inner member and output at least one position signal representing at least one of the linear position and angular position of the outer member relative to the inner member.

20. The system of claim 19, wherein the position detector includes a biasing device configured to bias the outer member toward a neutral position.

21. The system of claim 19, wherein the input device includes a ball joint disposed at a distal end of the inner member and configured to allow the outer member and inner member to rotate about an axis defined by the ball joint.
